(19) United States
(12) Patent Application Publication
(10) Pub. No.: US 2017/0113059 A1
(43) Pub. Date: Apr. 27, 2017

(54) Wrist worn sensor

(71) Applicant: WRISTSPACE, LLC, Mount Vernon, OH (US)

(72) Inventor: Richard John Fisher, Mount Vernon, OH (US)

(21) Appl. No.: 15/303,217

(22) PCT Filed: Apr. 10, 2015
(86) PCT No.: PCT/US15/25441

§ 371 Date: Oct. 10, 2016

Related U.S. Application Data
(60) Provisional application No. 62/032,059, filed on Aug. 1, 2014.
(60) Provisional application No. 61/978,766, filed on Apr. 11, 2014.

(51) Int. Cl. A61N 2/02; A61B 5/00; A61B 5/026; A61N 2/00; A61B 5/024

(52) U.S. Cl. CPC A61N 2/02; A61N 2/008; A61B 5/02416; A61B 5/0261; A61B 5/02405; A61B 5/6831

(57) ABSTRACT

The invention is a wrist worn Photoplethysmography Sensor (“PPG”) and Pulsed Electro Magnetic Field (“PEMF”) integrated into a thin ergonomic wristband. The invention will measure heart rate, oxygen saturation, and heart rate variability. In addition to PPG measurements, the PEMF generator treats a user's blood with an electromagnetic field tailored to the individual. The electromagnetic field washes red blood cells and/or erythrocytes and/or any other vertebrate organisms which principal means of delivering oxygen and other cellular nutrients to the body tissues via blood flow through the circulatory system for optimal CELLHEALTH™.

### CROSS-REFERENCE TO RELATED APPLICATION

This application claims priority to PCT application number PCT/US2015/025441 filed Apr. 10, 2015; U.S. provisional application No. 62/032,059 filed Aug. 1, 2014; and U.S. provisional application No. 61/978,766 filed Apr. 11, 2014 the contents of which are hereby incorporated by reference.

### BACKGROUND

The present invention relates to the measurement and treatment of individual users with a pulsed electromagnetic field (PEMF) generator integrated in to a band that can be worn on the wrist.

Traditional methods for treating certain injuries to a body (e.g., humans, animals) involve setting and immobilize the injured member to allow natural healing of the injury. Often, it is desirable that natural healing will restore damaged structures to their original uninjured condition without significant inconvenience to a patient. Traditional problems in injury treatments have been associated with the inability of the patient's body to heal correctly or rapidly and the inability of the healed part to regain fall strength and freedom of movement. These problems are especially acute in patients with suboptimal health and reduced healing capacity such as elderly, bedridden or patients with multiple disorders.

Attempts to address these problems and to promote more rapid healing have led to the use of pulsed electromagnetic fields. It is generally known that electromagnetic fields applied to a body can produce favorable biological effects. For example, FDA approved PEMF apparatuses are available for use in bone healing. These apparatuses are used to augment and accelerate the natural healing process. PEMF is also effective in the treatment of severe injuries and fractures which are not otherwise treatable using conventional techniques.

Known methods and apparatuses which have been used to treat injuries using PEMF include the use of Helmholtz and toroidal coils to deliver PEMF. These methods and apparatuses have suffered from various deficiencies. For example, Helmholtz coils suffer from field inhomogeneity and field dropouts (e.g., the field drops to zero near the center of the coil). Toroidal coils are inefficient and have a relatively weak field strength. Further, known methods of PEMF treatment have problems associated with system complexity, large size and weight, long treatment times, weak PEMF strength and low efficiencies in promoting healing. Current devices and methods of PEMF treatment further fail to provide adequate mobility during treatment. Other drawbacks also exist.

Physical fitness is known to benefit people in many different areas, including improved flexibility and range of motion, increased muscular strength and cardiovascular fitness, body fat loss and increased stamina. Physical exercise helps maintain good health, increases energy, reduces stress and improves physical appearance. However, in order to gain the benefits of regular physical exercise, the users need to be able to conveniently access PEMF therapy for continual cell health washing and information concerning their exercise level and receive a feedback concerning their fitness activity. By accessing PEMF therapy continually via a conveniently dawned wrist device during exercise users can benefit from the increase in cellular oxygen/nutrient transport for increased recovery times, increased cardiovascular/aerobic performance, reduced lactic acid build up among other benefits.

Conventional devices are known for providing such information to the users. For example, U.S. Pat. No. 5,810,722 describes a device for measuring heartbeat rate. An athlete or a person engaged in fitness training may wear the device on the breast or the wrist. The device measures the heartbeat rate based on skin contact and allows the user to read the result from a display provided in the casing of the device. U.S. Pat. No. 5,891,042 describes a fitness monitoring device which includes an electronic pedometer integrated together with a wireless heart rate monitor. The device may be secured to the user's belt or waist band. The device receives electrical signals from a telemetric transmitter unit arranged on the user's skin adjacent to his heart and calculates the heart rate. The device is also configured to detect the user's body motion at each step for performing step counting. The user can read the results from a display provided in the casing of the device. The display includes an alpha/numeric display portion and a heart rate monitoring icon. These prior art devices, however, merely allow the users to see the physiological information concerning their exercise level. They do not provide any processed feedback to the users. In addition, these devices can be cumbersome to wear and they force the users to monitor their own activity, thereby interfering with their focus on physical exercise.

This invention pertains generally to an apparatus and a method for in vitro and in vivo therapeutic and prophylactic treatment of plant, animal, and human tissue, organs, cells and molecules. In particular, an embodiment according to the present invention pertains to use of non-thermal time-varying magnetic fields configured for optimal coupling to target pathway structures such as molecules, cells, tissue, and organs, using power and amplitude comparison analysis to evaluate a signal to thermal noise ratio (“SNR”) in the target pathway structure. Another embodiment according to the present invention pertains to application of bursts of arbitrary waveform electromagnetic signals to target pathway structures such as molecules, cells, tissues, and organs using ultra lightweight portable coupling devices such as inductors and electrodes, and driver circuitry that can be incorporated into a positioning device such as knee, elbow, lower back, shoulder, foot, and other anatomical wraps, as well as apparel such as garments, footwear, and fashion accessories.

This invention may also pertain generally to an apparatus and a method for treatment of living tissues and cells by altering their interaction with their electromagnetic environment. This invention also relates to a method of modification of cellular repair, maintenance, and general behavior by application of encoded electromagnetic information. More particularly this invention relates to the application of surgically non-invasive coupling of highly specific electromagnetic signal patterns to the wrist. In particular, an embodiment according to the present invention pertains to using PEMF to enhance living tissue growth and repair via increased health and maintenance of the shape of red blood cells.

Recent clinical use of non-invasive pulsed radio frequency (“PRF”) at radio frequencies has used pulsed bursts of a 27.12 MHz sinusoidal wave, each pulse burst typically exhibiting a width of sixty five microseconds and having approximately 1,700 sinusoidal cycles per burst, and with various burst repetition rates.

Broad spectral density bursts of electromagnetic waveforms having a frequency in the range of one to one hundred megahertz (MHz), with 1 to 100,000 pulses per burst, and with a burst-repetition rate of 0.01 to 10,000 Hertz (Hz), are selectively applied to human, animal and plant cells, organs, tissues and molecules. The voltage-amplitude envelope of each pulse burst is a function of a random, irregular, or other like variable, effective to provide a broad spectral density within the burst envelope.

An embodiment according to the present invention tailors the pulse pattern to the user's current physiologic state because the PPG sensor is receiving data and a processor is calculating in real time. The real time feedback allows the PEMF generator to increase or decrease pulse power, pulse wave form, and pulse frequency

Therefore, a need exists for an apparatus and a method that more effectively supports and maintains red blood cell health.

A pulsed radio frequency (“PRF”) signal derived from a 27.12 MHz continuous sine wave used for deep tissue healing is known in the prior art of diathermy. A pulsed successor of the diathermy signal was originally reported as an electromagnetic field capable of eliciting a non-thermal biological effect in the treatment of infections. Subsequently, PRF therapeutic applications have been reported for the reduction of post-traumatic and post-operative pain and edema in soft tissues, wound healing, burn treatment, and nerve regeneration. The application of PRF for resolution of traumatic and chronic edema has become increasingly used in recent years. Results to date using PRF in animal and clinical studies suggest that edema may be measurably reduced from such electromagnetic stimulus

Waveforms are selected using a feedback system; when blood volume is increased, a processor calculates an output level for the PEMF generator. Signals comprise bursts of at least one of sinusoidal, rectangular, chaotic and random wave shapes have frequency content in a range of 0.01 Hz to 100 MHz at 1 to 100,000 bursts per second, with a burst duration from 0.01 to 100 milliseconds, and a burst repetition rate from 0.01 to 1000 bursts/second. Peak signal amplitude at a target pathway structure such as tissue, lies in a range of 1 μV/cm to 100 mV/cm. Preferably, the present invention comprises a 20 millisecond pulse burst, repeating at 1 to 10 burst/second and comprising 5 to 200 microsecond symmetrical or asymmetrical pulses repeating at 0.1 to 100 kilohertz within the burst. Fixed repetition rates can also be used between about 0.1 Hz and about 1000 Hz. An induced electric field from about 0.001 mV/cm to about 100 mV/cm is generated. The number of daily treatments may be programmed to vary on a predefined protocol.

### RELEVANT ART REFERENCES

U.S. patent application Ser. No. 13/801,789 filed by PILLA et al. describes an APPARATUS AND METHOD FOR ELECTROMAGNETIC TREATMENT using body worn PEMF devices. U.S. Pat. No. 6,132,362 issued to Tepper et al. describes PULSED ELECTROMAGNETIC FIELD STIMULATION THERAPY SYSTEM WITH BI-PHASIC COIL for the treatment of soft tissue. International Patent Application Number PCT/US2012/061352 filed by Martinez et al. discloses a PULSED ELECTROMAGNETIC FIELD DEVICE WITH ADHESIVE APPLICATIOR for treating injured tissue. International Patent Application Number PCT/US2005/042873 filed by Maladay et al. discloses a METHOD AND APPARATUS FOR THERAPEUTIC TREATMENT OF INFLAMATION AND PAIN WITH LOW FLUX DENSITY, STATIC-ELECTROMAGNETIC FIELDS.

### BRIEF DESCRIPTION OF THE DRAWINGS

FIG. 1 is a preferred embodiment with a wristband comprising an ulna ergonomic cutout and PPG sensor linked to a microprocessor and PEMF generator for sending a PEMF through a coil in the wristband.

FIG. 2 is a wristband with a raised PPG sensor.

FIG. 3 is a preferred embodiment showing an ulna notch in the wristband relative to common large veins.

FIG. 4 shows a wrist as a loop inside a typically shaped round wrist band to illustrate issues related to complementing the wrist anatomy.

FIG. 5 is a preferred embodiment of a wristband that incorporates a copper ribbon wrapped in magnetic material that is further encased in a polymer doped with anionic minerals.

FIG. 6 shows a preferred embodiment of the present invention on a left hand with a cephalic vein and further discloses skeletal description showing the radius and ulna.

FIG. 7 shows a preferred embodiment of the present invention with a dorsal locking, magnetic clasp.

### SUMMARY OF THE INVENTION

The present invention is a wristband with a PPG sensor in communication with a microprocessor. The microprocessor further controls a PEMF generator based on inputs from the PPG sensor. The wristband further comprises a power source and a communication means such as a Wi-Fi or Bluetooth transmitter to communicate with a mobile device. The mobile device is able to record, store, and manipulate the data from the wristband, and further capable of programing or adjusting the PMEF treatment. The wristband is particularly accurate because the integration of multiple sensors allows the microprocessor to compare at least two sets of data simultaneously and based on that the microprocessor can calculate the noise in the signal and subtract out the noise.

In one embodiment, personal data capturing functionality is integrated into a wireless communication device or a portable computing device by incorporating one or more personal parameter receivers into the wireless communication device or the portable computing device. In another embodiment, personal data capturing functionality is integrated into a wireless communication device or a portable computing device by attaching a personal data capture device to the wireless communication device or the portable computing device. The personal data capture device is configured to receive personal data of a user and transmit the personal data to the wireless communication device or the portable computing device, either of which is capable of transmitting the personal data to a network server over a wireless network.

Various improvements of the present invention may be apparent to those skilled in the art.

### DETAILED DESCRIPTION OF THE DRAWINGS

Referring now to the drawings wherein the showings are for purposes of illustrating a preferred embodiment of the present invention and not for purposes of limiting the same.

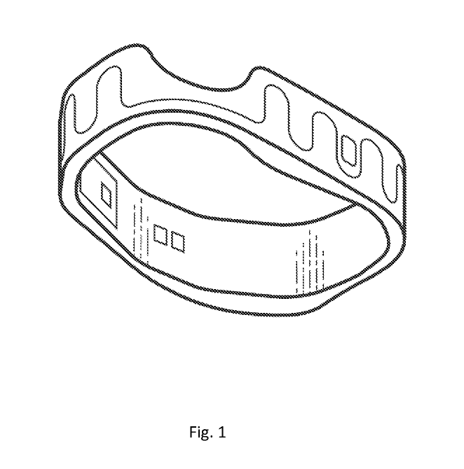

FIG. 1 shows a preferred embodiment 10 of the PEMF device that can run specific programs at various times of the day for optimized cell health. For example, the preferred embodiment 10 may run predetermined protocols when specific cell rejuvenation is optimal during specific time periods. A perfect example of optimized cell washing could happen when specific organs are in a cleansing and rejuvenation mode based on a user's circadian rhythm cycle. These biorhythms go back to principles of Chinese medicine and principles of the meridian cycles, wherein the body is known to cycle through twelve 2 hour cycles every day and night during which each meridian organ system becomes most active. The cycle is regarded as starting at 3:00 am when the liver time concludes and the body's energies turn outward in readying the body for the day by cleansing the lungs and the large intestine. Then, from 3:00 pm onward the energies flow back in to restore mode and maintain the body during rest and sleep.

The meridians and their functioning times are described below:

| Time | Meridian |
|---|---|
| 1:00 am-3:00 am | Liver |
| 3:00 am-5:00 am | Lung |
| 5:00 am-7:00 am | Large |
| | Intestine |
| 7:00 am-9:00 am | Stomach |
| 9:00 am-11:00 am | Spleen |
| 11:00 am-1:00 pm | Heart |
| 1:00 pm-3:00 pm | Small |
| | Intestine |
| 3:00 pm-5:00 pm | Bladder |
| 5:00 pm-7:00 pm | Kidney |
| 7:00 pm-9:00 pm | Circulation- |
| | Sex |
| 9:00 pm-11: pm | *Triple |
| | Warmer |
| 11:00 pm-1:00 am | Gall |
| | bladder |

FIG. 1 further discloses that the preferred embodiment 10 is a thin band that fits around a user's wrist. There is a deformation in a distal top edge of the band to accommodate a user's ulna base. Disposed with in the band is a PEMF generator that is connected to a coil for emitting the electromagnetic pulse. The coil is preferably disposed within the circumference of the entire band; however, an alternative embodiment could concentrate the coil to a portion of the wrist closest to large veins. The preferred embodiment 10 requires a power source such as a lithium ion battery. The battery could be rechargeable or disposable. Also in the preferred embodiment 10 there is a PPG sensor with LED capable of measuring a user's blood flow. The preferred embodiment 10 also may contain a microprocessor that calculates blood flow, pulse rate, oxygen flow, heart rate variability, detection of heart conditions at a sampling rate of up to 2000 per minute from data received from the PPG sensor. Based on the PPG sensor data the microprocessor can adjust the PEMF generator output frequency, power, wave form, pulse rate. The preferred embodiment 10 may also contain a Wi-Fi or Bluetooth enabled antenna for communication to a mobile device, like a smartphone, or a computer for a user to record and analyze the PPG sensor data. Additionally, in the preferred embodiment 10 the PPG sensor could incorporate a movement cancellation technique to reduce noise and improve accuracy of the overall system. In a preferred embodiment the cancellation technique uses the technology disclosed in U.S. patent application Ser. No. 13/974,758. Specifically, multiple sensors may be disposed in close proximity to each other. The differences in signal processing can be compared to subtract out noise and increase the accuracy of the microprocessor outputs. Additionally, the preferred embodiment 10 may include an accelerometer to aid in calculating movement of a user.

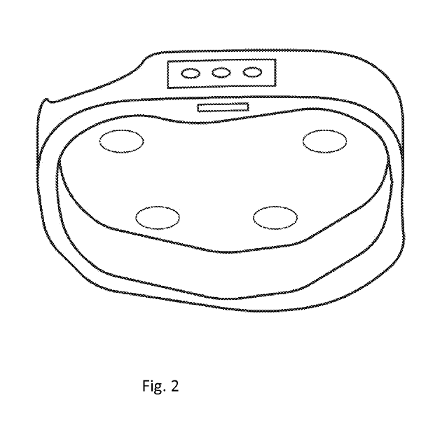

FIG. 2 discloses a preferred embodiment 10 wherein the cephalic vein and basilica vein show the orientation of the band relative to the median antebrachial vein. Most wrist devices like watches utilize a tourniquet approach that ratchets and hooks to lock the band in position. In particular, there is a need for a band that has constant tension but too much tension can compress the veins in the wrist and interfere with the sensitivity of the PPG sensor. Therefore, there is a need for the present invention to have portions of the band rigid and other portions semi-rigid so as to apply the least amount of compression to the wrist as possible while still maintaining a constant contact with the user's skin. For example, upper portion of the band that covers the top of the wrist and ulna base could be semi-rigid and the bottom portion of the band that covers the underside of the wrist could be rigid. In this configuration the compression would be furthest from compressing the targeted vein(s) for the PPG sensor(s). Additionally, too much pressure will reduce blood circulation due to capillary closure.

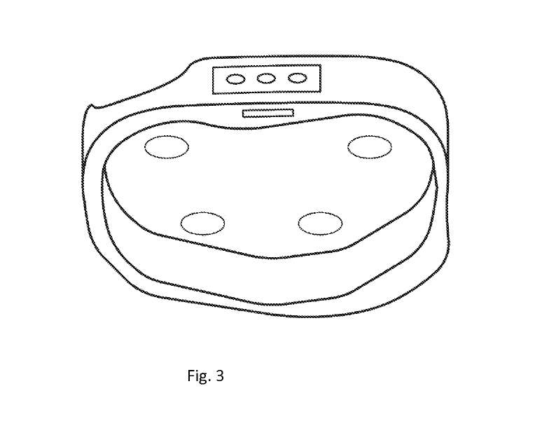

FIG. 3 illustrates an alternative preferred embodiment that incorporates a PPG sensor that is raised out of the inner surface of the band's circumference. This raised sensor configuration would allow the total tension in the band to be reduced while pushing the PPG sensor into contact with the skin at a constant pressure. Thus, in this configuration the PPG sensor stays in place while reducing the need to compress the band at a higher pressure. This configuration further allows the sensor to detect a user's physiologic condition in a more natural status without additional blood vessel compression.

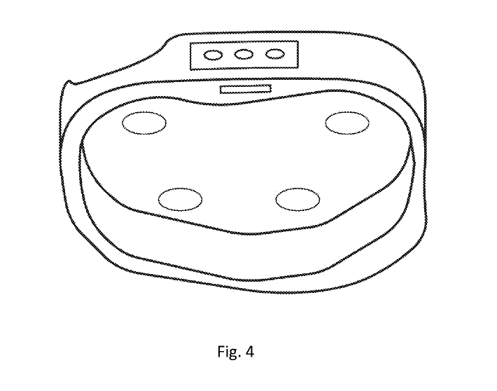

FIG. 4 shows a left hand dorsal view cross section of the wrist. An embedded accelerometer could count steps and calculate calories burned during a user's movement.

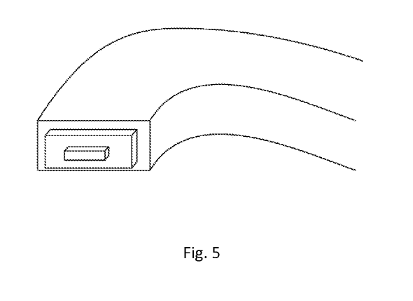

FIG. 5 Illustrates an alternative preferred embodiment 50 wherein the band is connected by a male and female magnetic connector. The male and female connector allows the user to tighten the band to the wrist and for the ability to produce a power loop that goes around the circumference of the wrist ensures a full 360 spectrum. The wristband has an outer polymer coating doped with anion particles. The polymer coating covers a magnetic material, for example, a soft material such as neodymium (NdFeB). Further, the wristband comprises a copper ribbon through the magnetic material. The band is composed of anion particles embedded in a silicone material. Additionally, the band will comprise material with higher durometer properties such as plastic, ABS, metal that will be relatively rigid at predetermined positions to complement anatomical contouring. For example at the ulna base. This produces a carrier frequency of up to 28 Megahertz, with a burst repetition of up to 2 milliseconds and an energy density 0.13±0.05 micro pulses Ws/cm.

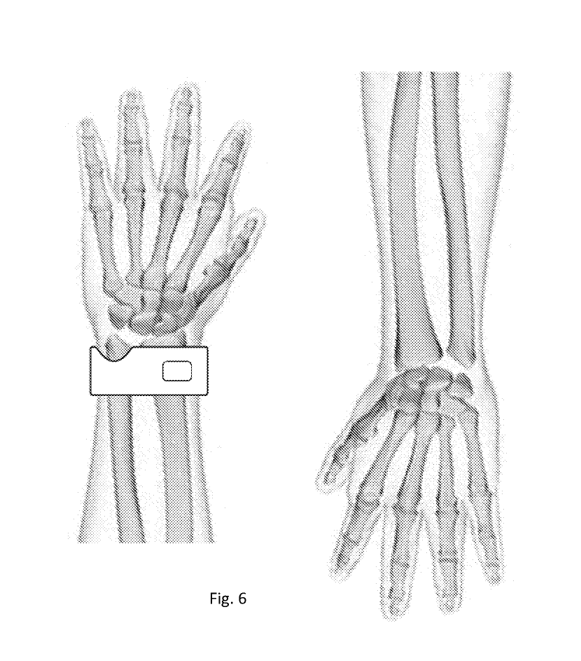

FIG. 6 illustrates a preferred embodiment on the left hand of a user. The distal or top edge of the preferred embodiment 60 has a curved shape to accommodate a typical human ulnar bone that typically projects upwardly and outwardly to the left of the left hand. The ulnar knob serves to keep the preferred embodiment 60 in place while a user is moving about. Specifically, the additional friction ensures that the preferred embodiment 60 does not slip in the axial direction without the need to increase compression of the preferred embodiment 60 which would artificially affect blood flow measurements. Additionally, this helps ensure alignment between the PEMF generator and the cephalic vein. Veins are vessels that carry de-oxygenated blood from the capillaries back to the heart. In human anatomy, blood flows from a variety of smaller veins, draining into the cephalic vein. This is specifically the large vein in the upper arm, running from the hand to the shoulder, along the outer edge of the biceps muscle. It passes between the deltoid and pectoralis major muscles, known as the deltopectoral groove, through the deltopectoral triangle and empties into the axillary vein. The large size of the vein, its visibility through the skin, and its reasonably consistent location in the deltopectoral groove makes it generally easy to insert large cannulae. Cannulae are used to drain fluid or to administer intravenous drugs. For this reason it is sometimes known as The Houseman's Friend. However, although the cephalic vein in the forearm is often used for intravenous catheters, its close proximity to the radial nerve sometimes causes it to be damaged when the vein is cannulated. Thus, the PEMF generator is located at the optimal position for efficient energy consumption to treat the user's blood.

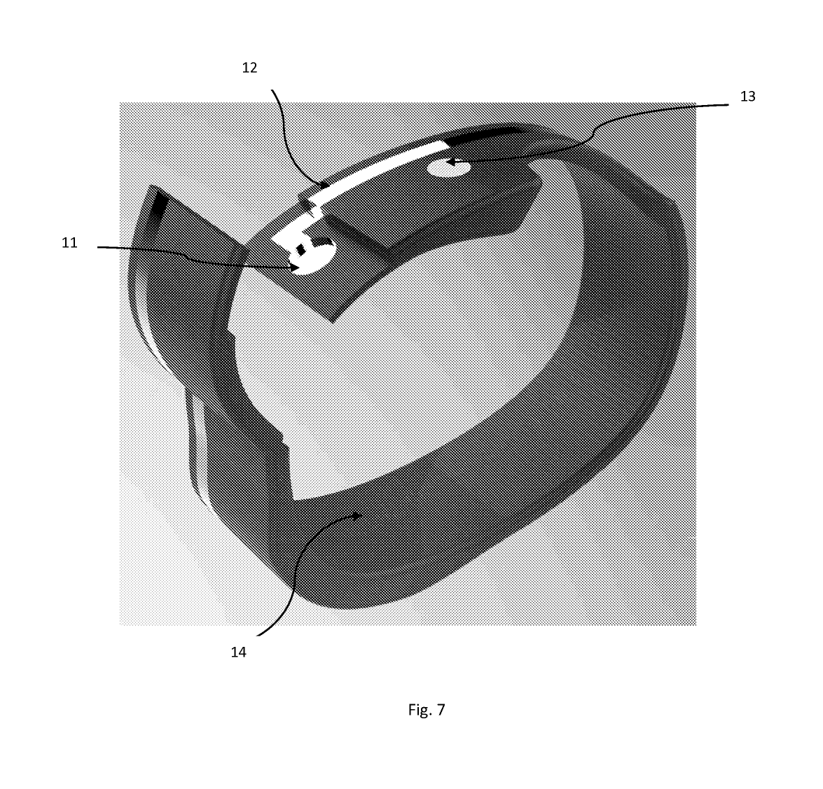

FIG. 7 illustrates a preferred embodiment, wherein the wristband closes by way of male and female 11 magnetic clasps, located on the dorsal surface of the wrist, when worn. A PEMF and transmitter 13 are also located along the dorsal surface of the wrist, when wearing the wristband; an ulnar notch is located adjacent to the transmitter and PEMF. A power source 14 is located ventral to the wrist when the wristband is worn. When the male and female clasps are connected to each other a circuit 12 is completed.

Another embodiment could be PEMF therapy activated automatically when the body's heart rate, pulse or accelerometer reaches a predetermined threshold. It could be determined that providing therapy when blood is flowing at its peak could potentially expose more cells to more therapy over shorter periods of time. It may also be hypothesized that the need could also exist for longer cell exposure to PEMF at a slow heart rate level. These device specifications may be altered and adjusted as determined by the user. The software platform within the wrist worn device will allow for the monitoring of various body signals through embedded sensor technology as well as adjustability of the PEMF therapy cycles and power output. The interface for monitoring, adjusting, tracking, downloading and reprogramming functions of the wrist worn device will be executed through a phone and/or computer “application”. The wrist worn PEMF device will have Bluetooth/WiFi programming capability that will allow the device to be sold as an Over-The-Counter, wrist worn PEMF generator for optimized CELLHEALTH. Additionally, On demand “Prescription Level” therapies may be unlocked via a phone or computer supported “application” by “Prescription Only” to work at higher power frequencies that fall within the scope of an FDA Class II or III medical device.

Typically the devices under product Code ILX are classified as Class III medical devices and deliver therapy in the form of 30 minute treatments with 8-12 hours between treatments.

A need exists for a device which delivers about 5 minutes of therapy at 1:00 am, 20 minutes at 9:00 am and 35 minutes starting at 9:35 pm for cell therapy. Unlike the current devices which focuses on localized therapy directly at the pain source, the current invention is focused on the cell washing of damaged cells that flow through the PEMF energy ring. Devices currently listed as product code ILX are indicated for adjunctive use in the palliative treatment of post-operative pain and edema in superficial soft tissue. The current invention is directed to improve blood cell health and overall rejuvenation of the blood.

An additional embodiment is a wristband that achieves contact with the skin with a raised PPG sensor mount. Thus, an elastic band will not compress tissue and create artifacts or interfere with the PPG sensor.

Additional embodiment is a dual censor system wherein differences in measurements between the two PPG sensors can be used to determine background noise and cancel artifacts to provide a more true analysis of the user's health.

In a preferred embodiment the width of the wristband is 15-30 mm, while the thickness may vary over various portions. For example, the coil embedded in the wristband is not required to be more than 3 mm thick. The PPG sensor portion may be from 3-10 mm depending on configurations. For example, if the PPG sensor is raised that would add thickness to that portion of the wristband. Also, it is contemplated that a battery could be accommodated up to 10 mm in thickness and yet keep the overall weight low for the user.

An alternative preferred embodiment adapts the PPG sensor to be printed directly on to the wristband inner surface. For example, there are many companies that perform flexible printing onto polyester substrates of 3 to 5 mil for flexible applications. The process includes load adhesives with carbon polymers and/or Silver-Silver Chloride materials for high conductivity circuitry. These Silver-Silver Chloride are able to clearly detect very low current potentials.

Additional modifications and improvements of the present invention may also be apparent to those skilled in the art. Thus, the particular combination of parts described and illustrated herein is intended to represent only one embodiment of the invention, and is not intended to serve as a limitation of alternative devices within the spirit and scope of the invention.

What is claimed:

1. A method for applying an electromagnetic field to a patient comprising the steps of: applying one or more therapeutic coil to an injured area of a patient's body; and conducting a periodic electric current to the one or more therapeutic coil to generate an electromagnetic field, wherein said one or more therapeutic coil comprises a core with an bottom surface and a top surface defining a thickness, a first side edge portion and a second side edge portion defining a height, a first end portion and second end portion defining a width and an electrically conductive wire wound a plurality of turns around the first end portion, the bottom surface, the second end portion and the top surface, to form a coil comprising a plurality of loops, each loop having at least two dimensions wherein a first dimension is at least about 5 times as great as a second dimension.

2. The method of claim 1 wherein the periodic electric current is a non-inverting current.

3. The method of claim 1 wherein the periodic electric current is selected from the group consisting of square waves, sawtooth waves, triangle waves, sinusoidal waves, and rectified sinusoidal waves.

4. The method of claim 1 wherein the periodic electric current is of a frequency which provides a non-inverting electromagnetic field.

5. The method of claim 1 wherein the periodic electric current comprise a plurality of pulses, each pulse comprising a half sine wave current with a duration of about 0.5 to about 2 milliseconds.

6. The method of claim 5, wherein the plurality of pulses is delivered at a rate of about 100 pulses to about 4000 pulses per second.

7. The method of claim 5, wherein the plurality of pulses is delivered at the cyclotron resonance frequency of a pre-selected ion.

8. The method of claim 7, wherein the ion is selected from the group consisting of copper, silver, gadolinium, potassium, chloride, zinc, cobalt, iron, manganese, sodium, calcium, magnesium and lithium ions.

9. The method of claim 1 wherein the injured area is a soft tissue injury.

10. The method of claim 1 wherein the step of applying further comprises the step of placing a plurality of electrically connected coils to the injury to optimize the magnetic field between coils at the injury.

11. The method of claim 1 further comprising the step of applying a static magnetic field to the injured area.

12. The method of claim 11 wherein the static magnetic field is supplied a permanently magnetized core or a constant current through the one or more therapeutic coil.

13. The method of claim 1, wherein a North pole of said electromagnetic field is positioned next to the injured area of a patient's body.

14. The method of claim 1, fisher comprising the step of applying heat in proximity to said injured area of a patient's body.

15. The method of claim 14 wherein said step of applying heat is accomplished using an electrical resistance heater.

16. The method of claim 14 wherein said step of applying heat is accomplished using a chemical heater.

17. A method for integrating personal data capturing functionality into a wireless communication device and for analyzing and supplying feedback information to a user, the method comprising: receiving personal data of said user by at least one personal parameter receiver, the personal data comprising step data corresponding to a number of steps counted during an activity of said user; capturing the personal data in the wireless communication device; periodically transmitting the personal data from the wireless communication device to a network server over a wireless network; at the network server, storing in a repository of personal data maintained by, or accessible from, the network server, the personal data from said user; at the network server, analyzing the personal data to generate feedback information for said user; at the network server, posting the feedback information to a web site that is accessible to said user; wherein said receiving, capturing, periodically transmitting, storing, analyzing and posting are performed with respect to personal data for each of a plurality of users received from their corresponding wireless communication devices, and wherein said analyzing further comprises comparing personal data for said user with personal data for at least one other different user from the received personal data from said plurality of users, and wherein posting comprises posting comparisons between the personal data of said user and personal data for said at least one other different user.

18. The method of claim 17 wherein the at least one personal parameter receiver is contained in a personal data capture device attachable to the wireless communication device.

19. The method of claim 17 wherein the at least one personal parameter receiver is contained in the wireless communication device.

20. The method of claim 17 wherein analyzing comprises analyzing the personal data according to health and/or fitness of said user such that the feedback information comprises information pertaining to health or fitness of said user.

21. The method of claim 17 wherein posting comprises posting the feedback information and the personal data in a form comprising one or more of: graphs, charts, tables and map overlays.

22. The method of claim 17 wherein analyzing further comprises generating for presentation to said user in the feedback information instructions from one or more of: a fitness instructor, physician, athletic trainer, or nutritionist.

23. The method of claim 17 wherein posting comprises posting the feedback information to a web site that is accessible by said plurality of users.

24. The method of claim 17 wherein posting comprises posting the feedback information and the personal data of said user to a personal web site of said user.

25. A method for integrating personal data capturing functionality into a portable computing device and for analyzing and supplying feedback information to a user, the method comprising: receiving personal data of said user by at least one personal parameter receiver, the personal data comprising step data corresponding to a number of steps counted during an activity of said user; capturing the personal data in the portable computing device; periodically transmitting the personal data from the portable computing device to a network server over a wireless network; at the network server, storing in a repository of personal data maintained by, or accessible from, the network server, personal data from said user; at the network server, analyzing the personal data to generate feedback information for said user; and at the network server, posting the feedback information to a web site that is accessible to said user; wherein said receiving, capturing, periodically transmitting, storing, analyzing and posting are performed with respect to personal data for each of a plurality of users received from their corresponding wireless communication devices, and wherein said analyzing further comprises comparing personal data for said user with personal data for at least one other different user from the received personal data from said plurality of users, and wherein posting comprises posting comparisons between the personal data of said user and personal data for said at least one other different user.

26. The method of claim 25 wherein the at least one personal parameter receiver is contained in a personal data capture device attachable to the portable computing device.

27. The method of claim 25 wherein the at least one personal parameter receiver is contained in the portable computing device.

28. The method of claim 25 wherein analyzing comprises analyzing the personal data according to health and/or fitness of said user such that the feedback information contains information pertaining to health or fitness of said user.

29. The method of claim 25 wherein posting comprises posting the feedback information and the personal data in a form comprising one or more of: graphs, charts, tables and map overlays.

30. The method of claim 25 wherein analyzing further comprises generating for presentation to said user in the feedback information instructions from one or more of: a fitness instructor, physician, athletic trainer and nutritionist.

31. The method of claim 25 wherein posting comprises posting the feedback information to a web site that is accessible by said plurality of users.

32. The method of claim 25, wherein posting comprises posting the feedback information and the personal data of said user to a personal web site of said user.

33. A low-power, and lightweight electromagnetic treatment device for treating a target tissue by stimulation of a target pathway structure, the device comprising: a lightweight applicator comprising a flexible wire loop that is bendable to conform to a target anatomy, wherein the applicator is configured to deliver an electromagnetic signal to a target tissue; and a control circuit including a micro-controller configured to control the burst duration, the burst period, and the duration of a single treatment application, wherein the micro-controller is configured to apply broad spectral density bursts of electromagnetic waveforms to achieve maximal signal power within a bandpass of a biological target; and wherein the control circuit further comprises a pulse phase timing control configured to regulate the burst duration and burst period and configured to provide a signal to the applicator to induce an electric field of amplitude of between about 1 ?V/cm to about 100 mV/cm at the target tissue and a peak induced magnetic field between about 1 ?T and about 20 ?T, wherein the control circuit generates a burst of waveforms having a burst duration of greater than 0.5 msec and a burst period of between about 0.1 to about 10 seconds to produce a signal that is above background electrical activity in the target tissue.

34. The device of claim 33, wherein the device is wearable.

35. The device of claim 33, wherein the waveforms generated by the control circuit have a frequency of approximately 27.12 MHz.

36. The device of claim 33 further comprising a therapeutic device for positioning said applicator in proximity to a target pathway structure.

37. The device of claim 41, wherein the therapeutic device includes at least one of an anatomical support, an anatomical wrap, apparel, a mattress, a mattress pad, a wheelchair, a therapeutic chair, a therapeutic bed, and sporting goods.

38. The device of claim 33, wherein the signal provided to the applicator is configured to capacitively couple to living cells and tissues to modulate angiogenesis and neovascularization for the treatment of cardiovascular diseases.

39. The device of claim 33, wherein the signal provided to the applicator by the control circuit is configured to inductively couple to living cells and tissues to modulate angiogenesis and neovascularization for the treatment of cardiovascular diseases.

40. The device of claim 33, wherein the signal provided to the applicator by the control circuit is inductively coupled to living cells and tissues to modulate angiogenesis and neovascularization for the treatment of cerebral diseases.

41. The device of claim 33, wherein the signal provided to the applicator by the control circuit is capacitively coupled to living cells and tissues to modulate angiogenesis and neovascularization for the treatment of cerebral diseases.

42. The device of claim 33, wherein the signal provided to the applicator by the control circuit is inductively coupled to living cells and tissues to modulate angiogenesis and neovascularization for the treatment of cerebrovascular disease.

43. The device of claim 33, wherein the signal provided to the applicator by the control circuit is capacitively coupled to living cells and tissues to modulate angiogenesis and neovascularization for the treatment of cerebrovascular disease.

44. The device of claim 33, further comprising a delivery means for standard physical therapy modalities.

45. The device of claim 49, wherein said standard physical therapy modalities includes heat, cold, massage, and exercise.

46. The device of claim 33, wherein the background electrical activity is baseline thermal fluctuations in voltage and electrical impedance at the target pathway structure.

47. The device of claim 33, further comprising a chest garment, wherein the device is incorporated into the chest garment.

48. The device of claim 33, wherein the applicator comprises at least one of an inductive coupling means and a capacitive coupling means, connected to the micro-controller for delivering an electromagnetic signal to a fibrous capsule formation and capsular contracture target pathway structure.

49. The device of claim 33, wherein the signal generated by the control circuit comprises a signal to noise ratio or power signal to noise ratio of at least about 0.2 to modulate ion and ligand interactions in a fibrous capsule formation and capsular contracture target pathway structure above baseline thermal fluctuations in voltage and electrical impedance at the fibrous capsule formation and capsular contracture target pathway structure.

50. The device of claim 33, wherein said a lightweight applicator is in the form of a band; wherein said band is connected by a male and female magnetic connector; wherein said male and female connector allows the user to tighten the band to the wrist; wherein said magnetic connector provides the ability to produce a power loop that goes around the circumference of the wrist ensures a full 360° spectrum; wherein said connection must be completed to generate a 6.0 Hz to 28.0 Hz pulsed frequency with a field strength measured in a Tesla coil; wherein said band has an outer polymer coating doped with anion particles; wherein a polymer coating covers said magnetic material.

51. The device of claim 50 wherein said band comprises a copper ribbon through the magnetic connector.

52. The device of claim 50, wherein said band is composed of anion particles embedded in a silicone material.

53. The device of claim 50, wherein said band comprises material with high durometer properties such as plastic, ABS, metal which are relatively rigid at predetermined positions to complement anatomical contouring.
